(19) United States
(12) Patent Application Publication
(10) Pub. No.: US 2026/0083425 A1
(43) Pub. Date: Mar. 26, 2026

(54) INFORMATION PROCESSING APPARATUS, METHOD, AND PROGRAM

(71) Applicant: FUJIFILM Corporation, Tokyo (JP)

(72) Inventors: Shinichi KOJIMA, Tokyo (JP); Kazuma YOKOI, Tokyo (JP); Isao TAKAHASHI, Tokyo (JP)

(73) Assignee: FUJIFILM Corporation, Tokyo (JP)

(21) Appl. No.: 19/336,406

(22) Filed: Sep. 22, 2025

(30) Foreign Application Priority Data
Sep. 26, 2024 (JP) 2024-167877

(51) Int. Cl. A61B 6/58; A61B 6/03; A61B 6/42; G01T 7/00

(52) U.S. Cl. CPC A61B 6/585; A61B 6/032; A61B 6/4241; G01T 7/005

(57) ABSTRACT

A processor acquires projection data for multiple energy bins by detecting radiation transmitted through a subject using a photon-counting detector. It identifies first matching calibration data corresponding to the energy spectrum of the projection data from among multiple pieces of calibration data for material discrimination, each representing energy spectra of combinations of predefined calibration members. The processor then determines the presence or absence of a singularity in the attenuation coefficient based on differences, for each energy bin, between attenuation coefficients from the projection data and the first calibration data. If a singularity is determined to be present, the processor specifies second matching calibration data, selected from among the plurality of calibration data, based on the energy spectrum of the projection data at the higher- or lower-energy side of the singularity.

### CROSS REFERENCE TO RELATED APPLICATIONS

The present application claims priority from Japanese Patent Application No. 2024-167877, filed on Sep. 26, 2024, the entire disclosure of which is incorporated herein by reference.

### BACKGROUND

### Technical Field

The present disclosure relates to an information processing apparatus, method, and program.

### Related Art

In a radiographic imaging apparatus such as a photon-counting X-ray computed tomography (CT) apparatus, a material discrimination technique is known that uses the fact that absorption characteristics of radiation differ depending on a material and discriminates materials contained in a subject by using data corresponding to a plurality of energy bins. By using such a material discrimination technique, it is possible to acquire a material discrimination image in which a specific material contained in the subject is discriminated, in addition to a normal CT image (refer to, for example, JP2024-032518A).

Meanwhile, an index representing the absorption of photons by a material (that is, an attenuation coefficient) generally tends to decrease continuously as the photon energy increases. However, in a material with a high atomic number (hereinafter referred to as a high atomic number material), it is known that there is a singularity such as a K absorption edge (also referred to as a K-edge), at which the attenuation coefficient changes discontinuously within an energy range of the measured X-rays. Hereinafter, a material that includes a K absorption edge within the energy range of the measured X-rays is referred to as a high atomic number material. Examples of such a high atomic number material include a contrast agent injected into the subject and artificial objects contained in a body of the subject (such as a gold dental restoration, a bolt for bone fixation, and an embolization coil for thrombosis). Material discrimination is based on the assumption that the attenuation coefficient of a material changes continuously. Therefore, in a case where a material whose attenuation coefficient changes discontinuously is contained in the subject, accurate material discrimination cannot be performed.

### SUMMARY OF THE INVENTION

The present disclosure has been made in view of the above-described circumstances, and an object of the present disclosure is to enable accurate material discrimination.

According to the present disclosure, there is provided an information processing apparatus comprising: a storage unit configured to store a plurality of pieces of calibration data for material discrimination, the plurality of pieces of calibration data being acquired by measuring a plurality of types of calibration members, each consisting of a combination of two or more types of base materials having different compositions, using a photon-counting detector that converts incident radiation into a detected photon count for each of a plurality of energy bins, the number of which is three or more, the plurality of pieces of calibration data representing energy spectra for combinations of the calibration members; and a processor, in which the processor is configured to: acquire projection data for each of the plurality of energy bins, the projection data being acquired by detecting radiation transmitted through a subject using the photon-counting detector; specify first matching calibration data that matches an energy spectrum of the projection data from among the plurality of pieces of calibration data; determine presence or absence of a singularity in an attenuation coefficient based on the projection data, based on a difference, for each of the plurality of energy bins, between an attenuation coefficient based on the first matching calibration data and the attenuation coefficient based on the projection data; and specify, in a case where determination is made that the singularity is present, second matching calibration data that matches the projection data from among the plurality of pieces of calibration data, based on an energy spectrum of the projection data on a higher-energy side or a lower-energy side of the singularity.

In the information processing apparatus according to the present disclosure, the processor may be configured to specify, using a maximum likelihood estimation method, calibration data that matches the energy spectrum of the projection data from among the plurality of pieces of calibration data.

In the information processing apparatus according to the present disclosure, the processor may be configured to determine that the singularity is present in a case where a difference, for each of the plurality of energy bins, between an attenuation coefficient based on the specified calibration data and the attenuation coefficient based on the projection data is equal to or greater than a predetermined threshold value.

In the information processing apparatus according to the present disclosure, the processor may be configured to: derive a material discrimination image based on the first matching calibration data in a case where determination is made that the singularity is not present; and derive a material discrimination image based on the second matching calibration data in a case where determination is made that the singularity is present.

According to the present disclosure, there is provided an information processing method in an information processing apparatus including a storage unit configured to store a plurality of pieces of calibration data for material discrimination, the plurality of pieces of calibration data being acquired by measuring a plurality of types of calibration members, each consisting of a combination of two or more types of base materials having different compositions, using a photon-counting detector that converts incident radiation into a detected photon count for each of a plurality of energy bins, the number of which is three or more, the plurality of pieces of calibration data representing energy spectra for combinations of the calibration members, the information processing method comprising: causing a computer to execute: acquiring projection data for each of the plurality of energy bins, the projection data being acquired by detecting radiation transmitted through a subject using the photon-counting detector; specifying first matching calibration data that matches an energy spectrum of the projection data from among the plurality of pieces of calibration data; determining presence or absence of a singularity in an attenuation coefficient based on the projection data, based on a difference, for each of the plurality of energy bins, between an attenuation coefficient based on the first matching calibration data and the attenuation coefficient based on the projection data; and specifying, in a case where determination is made that the singularity is present, second matching calibration data that matches the projection data from among the plurality of pieces of calibration data, based on an energy spectrum of the projection data on a higher-energy side or a lower-energy side of the singularity.

According to the present disclosure, there is provided an information processing program for causing a computer to function as an information processing apparatus including a storage unit configured to store a plurality of pieces of calibration data for material discrimination, the plurality of pieces of calibration data being acquired by measuring a plurality of types of calibration members, each consisting of a combination of two or more types of base materials having different compositions, using a photon-counting detector that converts incident radiation into a detected photon count for each of a plurality of energy bins, the number of which is three or more, the plurality of pieces of calibration data representing energy spectra for combinations of the calibration members, the information processing program causing the computer to execute: a procedure of acquiring projection data for each of the plurality of energy bins, the projection data being acquired by detecting radiation transmitted through a subject using the photon-counting detector; a procedure of specifying first matching calibration data that matches an energy spectrum of the projection data from among the plurality of pieces of calibration data; a procedure of determining presence or absence of a singularity in an attenuation coefficient based on the projection data, based on a difference, for each of the plurality of energy bins, between an attenuation coefficient based on the first matching calibration data and the attenuation coefficient based on the projection data; and a procedure of specifying, in a case where determination is made that the singularity is present, second matching calibration data that matches the projection data from among the plurality of pieces of calibration data, based on an energy spectrum of the projection data on a higher-energy side or a lower-energy side of the singularity.

The technology of the present disclosure may be applied to a program product.

According to the present disclosure, accurate material discrimination can be performed.

### BRIEF DESCRIPTION OF THE DRAWINGS

FIG. 1 is a schematic configuration diagram of a medical image capturing system comprising an information processing apparatus according to an embodiment of the present disclosure.

FIG. 2 is a diagram showing a hardware configuration of the information processing apparatus according to the present embodiment.

FIG. 3 is a diagram showing a functional configuration of the information processing apparatus according to the present embodiment.

FIG. 4 is a diagram illustrating a method of acquiring calibration data.

FIG. 5 is a diagram illustrating derivation of a difference between an attenuation coefficient based on first matching calibration data and an attenuation coefficient based on projection data.

FIG. 6 is a diagram showing a table of calibration data for illustrating derivation of the attenuation coefficient based on the projection data.

FIG. 7 is a diagram showing an attenuation coefficient of a high atomic number material.

FIG. 8 is a diagram illustrating derivation of a difference between an attenuation coefficient based on first matching calibration data and an attenuation coefficient based on projection data in a case where a singularity is present.

FIG. 9 is a diagram illustrating specification of second matching calibration data in a case where the singularity is present.

FIG. 10 is a flowchart showing processing performed in the present embodiment.

### DETAILED DESCRIPTION

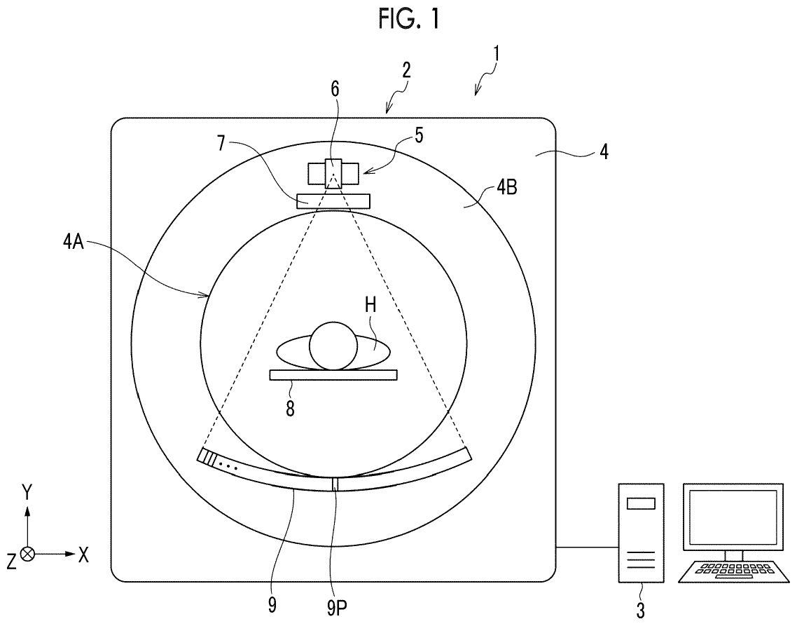

An embodiment of the present disclosure will be described in detail below with reference to the accompanying drawings. First, an example of a configuration of a medical image capturing system comprising an information processing apparatus of the present embodiment will be described. FIG. 1 is a schematic configuration diagram of the medical image capturing system comprising the information processing apparatus of the present embodiment.

A medical image capturing system 1 of the present embodiment comprises a CT apparatus 2 and a console 3, as shown in FIG. 1. The CT apparatus 2 comprises a gantry 4 and a patient table 8. In the following description, a horizontal direction in FIG. 1 is referred to as an X-axis, a vertical direction is referred to as a Y-axis, and a direction orthogonal to an XY plane is referred to as a Z-axis.

The gantry 4 has an opening portion 4A, and a subject H to be imaged is disposed within the opening portion 4A while being placed on the patient table 8. The gantry 4 and the patient table 8 are configured to move relative to each other in a Z-axis direction.

Inside the gantry 4, a radiation source 5 including a radiation tube 6 and a bowtie filter 7, and a detector 9 are disposed to face each other with the subject H interposed therebetween. The bowtie filter 7 optimizes an exposure dose by increasing the dose near a center and reducing the dose in the peripheral areas, in order to suppress the exposure dose in peripheral portions. Radiation emitted from the radiation tube 6 is shaped by the bowtie filter 7 into a beam shape suitable for a size of the subject H and is then emitted to the subject H. The detector 9 detects the radiation that has been transmitted through the subject H, and generates projection data corresponding to a photon count of the detected radiation. As one example, the detector 9 of the present embodiment is a photon-counting detector in which a plurality of detection elements 9P that detect photon energy, which is the energy of photons of incident radiation, are disposed in an arc shape centered on a focal point of the radiation tube 6. In the present embodiment, the detector 9 detects the photon energy of incident radiation by dividing the photon energy of incident radiation into a plurality of energy bins.

It should be noted that, in the present embodiment, X-rays are used as an example of the radiation, but the present disclosure is not limited to this, and y-rays or the like may also be used.

The radiation source 5 and the detector 9 are rotated around the subject H by a rotation drive unit (not shown) of the gantry 4. As the radiation irradiation from the radiation source 5 and the detection of the radiation by the detector 9 are repeatedly performed in conjunction with the rotation of the radiation source 5 and the detector 9, data (hereinafter referred to as projection data) regarding the subject H is acquired for each radiation projection path. The projection data acquired by the detector 9 is output to the console 3 and stored in a storage of the console 3. A value of the data corresponding to each detection element 9P, which is a minimum unit of the projection data, is the count of photons detected by the detection element 9P. The projection data is acquired individually for each of the energy bins.

The dose of the radiation emitted from the radiation source 5, a rotation speed of the gantry 4, a relative movement speed between the gantry 4 and the patient table 8, and the like are set by the console 3 based on acquisition conditions for acquiring projection data, which are input by a user such as a technologist.

The console 3 of the present embodiment performs control related to acquisition of projection data, generation of medical images, control related to material discrimination, and the like. The console 3 is an example of the information processing apparatus of the present disclosure.

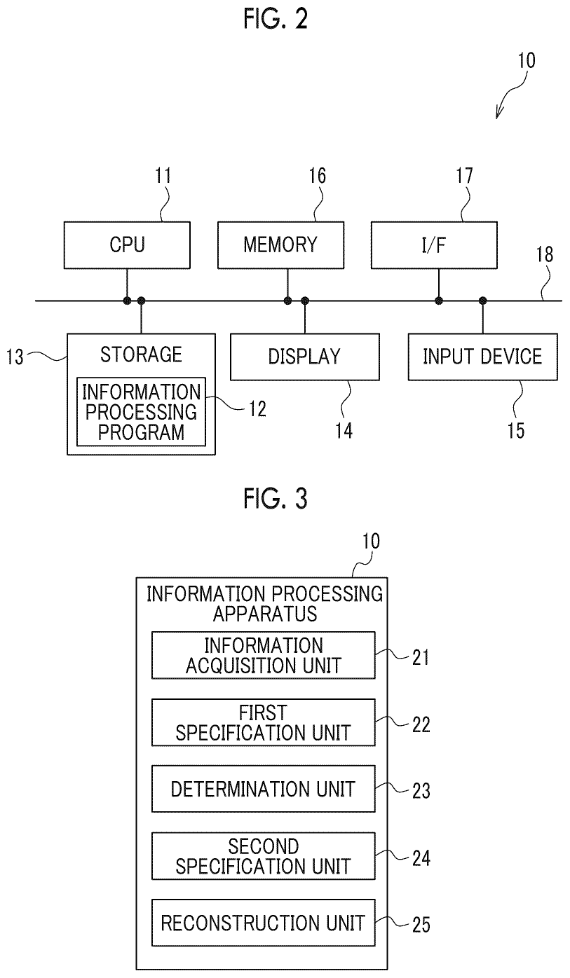

Next, the information processing apparatus according to the present embodiment will be described. First, a hardware configuration of the information processing apparatus according to the present embodiment will be described with reference to FIG. 2. As shown in FIG. 2, an information processing apparatus 10 is a computer, such as a workstation, a server computer, and a personal computer, and comprises a central processing unit (CPU) 11, a non-volatile storage 13, and a memory 16 as a temporary storage area.

In addition, the information processing apparatus 10 comprises a display 14, an input device 15, and an interface (I/F) 17. The CPU 11, the storage 13, the display 14, the input device 15, the memory 16, and the I/F 17 are connected to a bus 18. The CPU 11 is an example of a processor in the present disclosure.

The storage 13 is implemented using a hard disk drive (HDD), a solid-state drive (SSD), a flash memory, or the like. An information processing program 12 installed in the information processing apparatus 10 is stored in the storage 13 as a storage medium. The CPU 11 reads the information processing program 12 from the storage 13, loads the information processing program 12 into the memory 16, and executes the loaded information processing program 12. Additionally, the storage 13 stores calibration data, which will be described below. The storage 13 is an example of a storage unit of the present disclosure.

The display 14 is a device that displays various screens, and is, for example, a liquid crystal display or an electro luminescence (EL) display.

The input device 15 is used by the user to input scan conditions for acquiring projection data, instructions related to generation, display, and the like of images, various kinds of information, and the like. Examples of the input device 15 include various switches, buttons, a touch panel, a touch pen, a keyboard, a mouse, and the like. The display 14 and the input device 15 may be integrated into a touch panel display.

The I/F 17 performs communication of various kinds of information with the rotation drive unit (not shown) of the gantry 4, the radiation source 5, and the detector 9 via wired communication or wireless communication.

The information processing program 12 is stored in a storage device of a server computer connected to a network or in a network storage in a state accessible from the outside and is downloaded to and installed in a computer that constitutes the information processing apparatus 10 in response to a request. Alternatively, the information processing program 12 is distributed by being recorded on a recording medium such as a digital versatile disc (DVD) or a compact disc read-only memory (CD-ROM), and is installed in a computer that constitutes the information processing apparatus 10 from the recording medium.

Next, a functional configuration of the information processing apparatus according to the present embodiment will be described. FIG. 3 is a diagram showing the functional configuration of the information processing apparatus according to the present embodiment. As shown in FIG. 3, the information processing apparatus 10 comprises an information acquisition unit 21, a first specification unit 22, a determination unit 23, a second specification unit 24, and a reconstruction unit 25. The CPU 11 executes the information processing program 12 to function as the information acquisition unit 21, the first specification unit 22, the determination unit 23, the second specification unit 24, and the reconstruction unit 25.

The information acquisition unit 21 receives projection data P0 and calibration data C0 from the CT apparatus 2 via the I/F 17. The calibration data C0 is acquired by performing calibration of the detector 9 using a calibration member. Hereinafter, calibration will be described.

In the medical image capturing system 1 comprising the detector 9 which is a photon-counting detector, the projection data of the subject H for each of the energy bins (that is, an energy spectrum for each projection path) can be acquired. Therefore, a material discrimination image in which materials having different compositions are separated, and a medical image divided into a plurality of energy components can be generated. In order to obtain the material discrimination image and the like in this way, it is necessary to acquire calibration data C0 representing a relationship between the output in a case where combinations of a plurality of base materials, which are materials having known compositions, are measured by the detector 9 and the photon energy. Calibration refers to acquiring such calibration data C0.

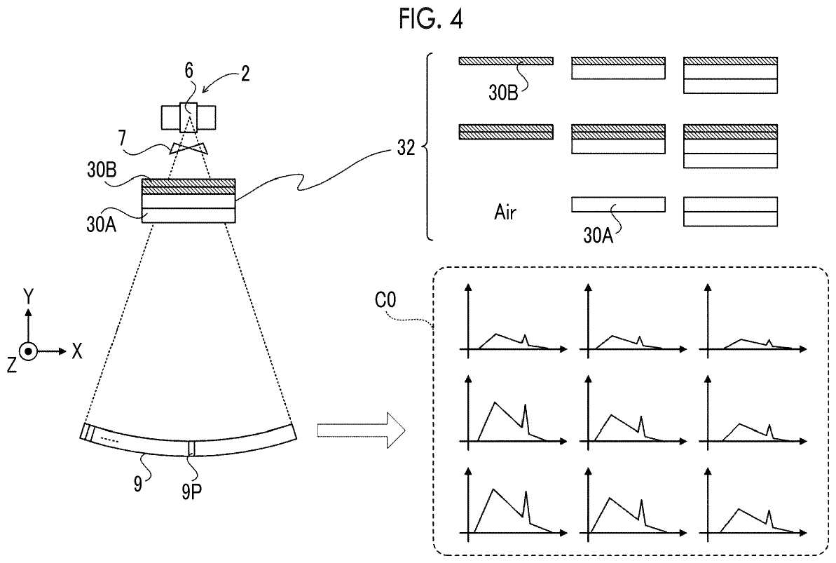

Hereinafter, an example of a method of acquiring the calibration data C0 will be described. FIG. 4 is a diagram illustrating the method of acquiring the calibration data. In order to acquire the calibration data C0, a calibration member consisting of a combination of one or more base materials having known compositions is used. In FIG. 4, a calibration member 30 consists of a combination of two types of base materials, that is, a first base material 30A and a second base material 30B. The first base material 30A and the second base material 30B have different attenuation coefficients with respect to radiation. In the present embodiment, the second base material 30B has a greater attenuation coefficient than that of the first base material 30A. Examples of the first base material 30A include water (soft tissue equivalent material), and examples of the second base material 30B include bone, which has a greater attenuation coefficient than that of water. Since it is difficult to measure water and bone, a water equivalent material is used as the first base material 30A, and a bone equivalent material or the like is used as the second base material 30B.

In the example shown in FIG. 4, a combination of two unit-thickness sheets of the first base material 30A and two unit-thickness sheets of the second base material 30B is used. In this way, the calibration data C0 is obtained for each of combinations 32 of thicknesses of the first base material 30A and the second base material 30B in a transmission direction of the radiation. For example, in a case where the thickness of the first base material 30A has M types and the thickness of the second base material 30B has N types, M×N pieces of calibration data C0 are obtained from Mx N types of combinations 32 of the base materials.

Specifically, in the example shown in FIG. 4, in a case where the calibration member 30 that does not use the first base material 30A is regarded as a calibration member 30 having a thickness of “zero” for the first base material 30A, the thickness of the first base material 30A has M=three types. Similarly, in a case where the calibration member 30 that does not use the second base material 30B is regarded as a calibration member 30 having a thickness of “zero” for the second base material 30B, the thickness of the second base material 30B has N=three types. Accordingly, in this case, there are nine types of calibration members obtained by combining the base materials, that is, 3×3=9 types. In FIG. 4, “Air” corresponds to the calibration member 30 that does not use either the first base material 30A or the second base material 30B, that is, the calibration member 30 in which the thickness of each of the first base material 30A and the second base material 30B is “zero”.

In the present embodiment, for each of the nine types of combinations 32, radiation is emitted from the radiation source 5, and radiation that has been transmitted through the combinations 32 is detected by the detector 9, whereby, for each combination 32, a photon energy spectrum (that is, a relationship between the energy of radiation and the photon count) is acquired as the calibration data C0. The nine types of calibration data C0 acquired in this manner are output to the console 3.

In the console 3, the calibration data C0 acquired from the CT apparatus 2 is stored in the storage 13 in association with the type of combination 32 used to acquire the calibration data C0. The stored calibration data C0 is used for material discrimination using the projection data P0 of the subject H.

In the present embodiment, four energy bins are set in the detector 9. Therefore, for each of the nine types of combinations 32 of the base materials 30A and 30B mentioned above, the calibration data C0 may be stored as a table representing the photon count for each of the four energy bins. Additionally, the calibration data C0 may be represented by a graph, a mathematical formula, or the like and stored in the storage 13.

The information processing apparatus 10 according to the present embodiment derives the material discrimination image by performing material discrimination using the projection data P0 acquired by the CT apparatus 2 imaging the subject H. For this purpose, the first specification unit 22 specifies first matching calibration data C1 that matches the energy spectrum of the projection data P0 from among a plurality of pieces of calibration data C0. Hereinafter, material discrimination will be described.

The projection data P0 is acquired at various projection angles in the CT apparatus 2 and has an energy spectrum of radiation for each of the detection elements 9P provided in the detector 9. The first specification unit 22 specifics, from among the energy spectra represented by the pieces of calibration data C0, the first matching calibration data C1 whose shape is closest to the energy spectrum of each piece of projection data P0 for each of the detection elements 9P, and acquires a combination of thicknesses of base materials corresponding to the specified energy spectrum. The estimation of the thicknesses of the base materials using such a method corresponds to performing fitting using a maximum likelihood estimation method between the energy spectrum represented by the calibration data C0 and the energy spectrum for each of the detection elements 9P.

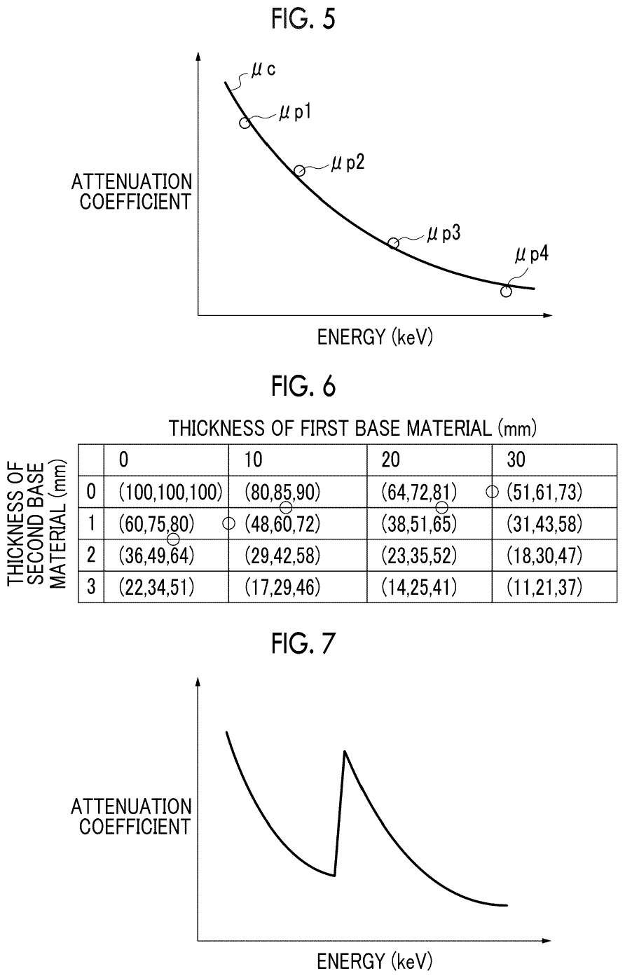

The determination unit 23 derives a difference between an attenuation coefficient uc based on the first matching calibration data C1 specified by the first specification unit 22 and an attenuation coefficient up based on the projection data P0. Then, the determination unit 23 determines the presence or absence of a K-edge in the attenuation coefficient up based on the projection data P0, based on the difference. FIG. 5 is a diagram illustrating the derivation of the difference between the attenuation coefficient uc based on the first matching calibration data C1 and the attenuation coefficient up based on the projection data P0. In FIG. 5, the horizontal axis represents the energy of radiation (keV), and the vertical axis represents the attenuation coefficient. In FIG. 5, the attenuation coefficient uc based on the first matching calibration data C1 is indicated by a solid line. The attenuation coefficient uc can be derived for each of the energy bins of the detection element 9P using a relationship of (the count with the calibration member)/(the count of Air)=exp (−μc(E)x), where E represents the energy of radiation, and x represents the thickness of the calibration member. Then, the derived attenuation coefficient for each of the energy bins can be derived by interpolation.

Meanwhile, the attenuation coefficient up based on the projection data P0 is derived as follows. FIG. 6 is a diagram showing a table of calibration data for illustrating the derivation of the attenuation coefficient up based on the projection data P0. Here, for the sake of description, it is assumed that the energy bins of the detection element 9P are three. In addition, it is assumed that the thickness of the first base material 30A is 0 mm (Air), 10 mm, 20 mm, and 30 mm, and the thickness of the second base material 30B is 0 mm (Air), 1 mm, 2 mm, and 3 mm. In each column of the table shown in FIG. 6, the photon counts in the three energy bins are shown in order from the lower-energy side.

Here, assuming that the counts in the respective energy bins of the projection data P0 are (58, 68, 78), the counts for the combinations of (0 mm, 0 mm), (10 mm, 0 mm), (20 mm, 0 mm), and (0 mm, 1 mm), among the combinations of thicknesses of the first base material 30A and the second base material 30B, are greater than those of the projection data P0, and the counts for the other combinations of thicknesses are smaller than those of the projection data P0. Therefore, in the table shown in FIG. 6, it can be understood that there are combinations of the first base material 30A and the second base material 30B, which correspond to the thickness of the subject H from which the projection data P0 has been acquired, at boundaries marked with circles between the columns of (0 mm, 0 mm), (10 mm, 0 mm), (20 mm, 0 mm), and (0 mm, 1 mm) and the other columns.

At these boundaries, the thicknesses of the first base material 30A and the second base material 30B are changed, and the counts in the respective energy bins corresponding to the changed thicknesses of the first base material 30A and the second base material 30B are derived by an interpolation operation. Then, a combination of thicknesses of the first base material 30A and the second base material 30B in which an error between the derived counts in the respective energy bins and the counts in the corresponding energy bins of the projection data P0 is minimized is derived. In this case, a combination of thicknesses of the first base material 30A and the second base material 30B in which the sum of squared differences in the counts in the corresponding energy bins is minimized need only be derived.

Based on the combination of thicknesses of the first base material 30A and the second base material 30B derived in this manner, the thickness of the subject H at the position of the detection element 9P from which the projection data P0 has been acquired is derived, and the attenuation coefficient up of the projection data P0 for each of the energy bins of the detection element 9P need only be derived using a relationship of (the count of the projection data P0)/(the count of Air)=exp (−μp(E)x), where E represents the energy of radiation, and x represents the derived thickness of the subject H.

Here, in a case where the high atomic number material is not contained in the body of the subject H or in a case where the high atomic number material is contained but radiation detected by the detection element 9P has not transmitted through the high atomic number material, when the attenuation coefficient μp based on the projection data P0 is plotted for each of the energy bins, the attenuation coefficient μp based on the projection data P0 substantially matches the attenuation coefficient uc of the first matching calibration data C1, as shown in FIG. 5. In the present embodiment, since the detection element 9P has four energy bins, in FIG. 5, plots are shown at four locations corresponding to attenuation coefficients μp1 to μp4.

In this case, a difference between the attenuation coefficient μc of the first matching calibration data C1 and the attenuation coefficient μp of the projection data P0 is smaller than a predetermined threshold value Th1. The difference need only be derived, for example, using the sum of squared differences in the counts in the corresponding energy bins. Therefore, the determination unit 23 determines that the K-edge is not present in the attenuation coefficient μp based on the projection data P0. In such a case, the thickness of the water and the thickness of the bone are obtained for each of the detection elements 9P in the projection data P0. Further, the projection data of the water and the projection data of the bone are obtained. Then, as will be described below, the reconstruction unit 25 can derive the material discrimination image by reconstructing a tomographic image for each base material from the plurality of pieces of obtained projection data for each base material.

On the other hand, as shown in FIG. 7, the attenuation coefficient of the high atomic number material exhibits a discontinuous change referred to as a K-edge. In this way, the energy spectrum of the projection data P0 for the material in which the K-edge is observed in the attenuation coefficient is unlikely to match any of the energy spectra of the plurality of pieces of calibration data C0. However, the first specification unit 22 specifies the first matching calibration data C1 that matches, that is, most closely matches, the energy spectrum of the projection data P0.

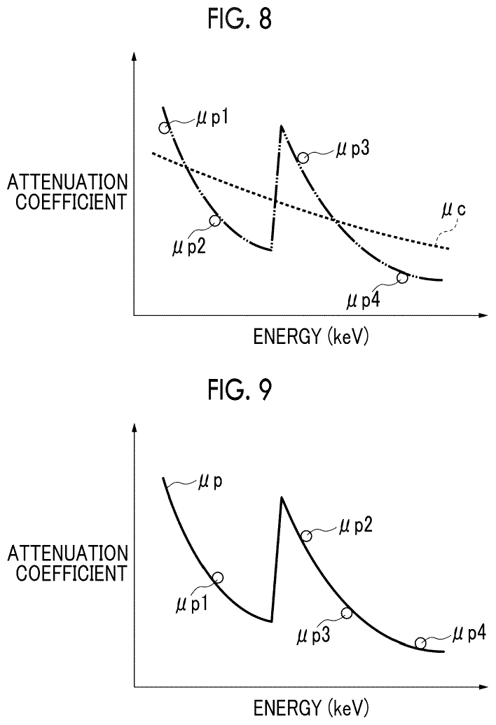

In such a case, in a case where the attenuation coefficients μp1 to μp4 based on the projection data P0 are plotted with respect to the attenuation coefficient μc based on the specified first matching calibration data C1, as shown in FIG. 8, the difference between the attenuation coefficient μc based on the first matching calibration data C1 and the attenuation coefficients μp1 to up4 based on the projection data P0 is large due to the presence of the K-edge. As a result, the difference between the attenuation coefficient μc and the attenuation coefficients μp1 to up4 is equal to or greater than the threshold value Th1. In this case, the determination unit 23 determines that the K-edge is present in the attenuation coefficient μp based on the projection data P0. In FIG. 8, the attenuation coefficient μp including the K-edge, which is assumed from the four attenuation coefficients μpl to up4 based on the projection data P0, is indicated by a virtual line.

In a case where an affirmative determination is made by the determination unit 23, the second specification unit 24 specifies second matching calibration data C2 that matches the projection data P0 from among the plurality of pieces of calibration data C0 based on an energy spectrum of the projection data P0 on a higher-energy side or a lower-energy side of the K-edge, which is the singularity. In this case, the second specification unit 24 estimates the K-edge from the attenuation coefficients μp1 to μp4 for the four energy bins based on the projection data P0. Then, the second matching calibration data C2 is specified using the energy spectrum on the side of the K-edge in the energy spectrum of the projection data P0, either the higher-energy side or the lower-energy side, where the number of energy bins is greater.

For example, as shown in FIG. 9, in the attenuation coefficient μp including the K-edge, which is assumed from the four attenuation coefficients μp1 to μp4, it is assumed that there are three energy bins on the higher-energy side of the K-edge. In this case, the second specification unit 24 specifies the second matching calibration data C2 by performing fitting with the plurality of pieces of calibration data C0 using the energy spectrum on the higher-energy side of the K-edge in the energy spectrum of the projection data P0. In a case where there are three energy bins on the lower-energy side of the K-edge, the second specification unit 24 specifies the second matching calibration data C2 by performing fitting with the plurality of pieces of calibration data C0 using the energy spectrum on the lower-energy side of the K-edge in the energy spectrum of the projection data P0.

In a case where the number of energy bins on the higher-energy side and the number of energy bins on the lower-energy side of the K-edge are equal, the second specification unit 24 compares the total photon count in the energy bin on the higher-energy side with the total photon count in the energy bin on the lower-energy side. Then, the second specification unit 24 derives the second matching calibration data C2 using the energy spectrum on the side having a greater total count.

In a case where an affirmative determination is made by the determination unit 23, and the second matching calibration data C2 is specified by the second specification unit 24, the thickness of the water and the thickness of the bone are obtained for each of the detection elements 9P in the projection data P0, in the same manner as in the above-described first matching calibration data C1. Further, the projection data of the water and the projection data of the bone are obtained. Then, the reconstruction unit 25 can derive the material discrimination image by reconstructing a tomographic image for each base material from the plurality of pieces of obtained projection data for each base material.

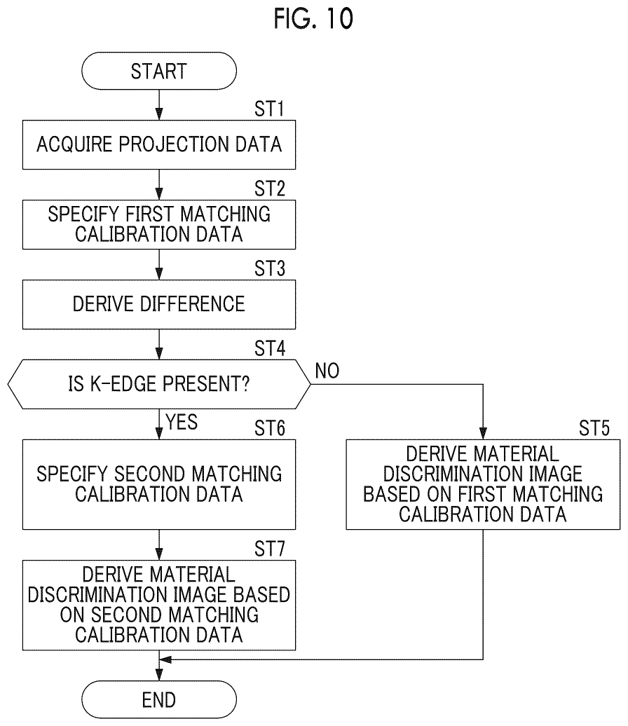

Next, processing performed in the present disclosure will be described. FIG. 10 is a flowchart showing the processing performed in the present embodiment. It is assumed that the calibration data C0 is acquired in advance and stored in the storage 13. First, the information acquisition unit 21 acquires the plurality of pieces of projection data P0 derived by imaging the subject H in the CT apparatus 2 (step ST1). Next, the first specification unit 22 specifies the first matching calibration data C1 that matches the energy spectrum of the projection data P0 from among the plurality of pieces of calibration data C0 (step ST2).

The determination unit 23 derives the difference between the attenuation coefficient μc based on the first matching calibration data C1 specified by the first specification unit 22 and the attenuation coefficient μp based on the projection data P0 (step ST3). Then, the determination unit 23 determines the presence or absence of the K-edge in the attenuation coefficient μp based on the projection data P0, based on the difference (step ST4).

In a case where a negative determination is made in step ST4, the reconstruction unit 25 derives the material discrimination image by reconstructing the tomographic image for each base material from the plurality of pieces of projection data for each base material obtained based on the first matching calibration data C1 (step ST5), and the processing ends.

In a case where an affirmative determination is made in step ST4, the second specification unit 24 specifies the second matching calibration data C2 that matches the projection data P0 from among the plurality of pieces of calibration data C0 based on the energy spectrum of the projection data P0 on the higher-energy side or the lower-energy side of the singularity (step ST6). The reconstruction unit 25 derives the material discrimination image by reconstructing the tomographic image for each base material from the plurality of pieces of projection data for each base material obtained based on the second matching calibration data C2 (step ST7), and the processing ends.

In this way, in the present embodiment, the first matching calibration data C1 that matches the energy spectrum of the projection data P0 is specified from among the plurality of pieces of calibration data C0, and the presence or absence of a singularity, such as the K-edge, in the attenuation coefficient μp based on the projection data P0 is determined based on the difference between the attenuation coefficient μc based on the specified first matching calibration data C1 and the attenuation coefficient μp based on the projection data P0. Then, in a case where determination is made that the singularity is present, the second matching calibration data C2 that matches the projection data P0 is specified from among the plurality of pieces of calibration data C0 based on the energy spectrum of the projection data P0 on the higher-energy side or the lower-energy side of the singularity.

Therefore, even in a case where a high atomic number material having a singularity in the attenuation coefficient is contained in the subject, and the projection data is acquired through transmission of that material, calibration data can be appropriately specified, and as a result, accurate material discrimination can be performed.

It should be noted that, in the above-described embodiment, four energy bins are set for the detector 9, but the number of bins is not limited to this. Any number of energy bins of three or more can be set. The number of energy bins is preferably three or more and eight or less because effective material discrimination cannot be performed in a case where the number of energy bins is too small, and the amount of calculation for the material discrimination increases in a case where the number of energy bins is too large.

In the present embodiment, each process of the information processing apparatus 10 is executed by any computer. In addition, any computer may execute these processes by means of a processor as hardware, a program as software, or a combination thereof. In that case, the processor is configured to execute various types of processing in the information processing apparatus 10 of the present embodiment in cooperation with the program and can function as each unit or each means in the present embodiment. Additionally, the execution order of the process by the processor is not limited to the order described above and may be changed as appropriate. Any computer may be a general-purpose computer, a computer for a specific application, a workstation, or another system capable of executing each process.

The processor may be configured using one or more pieces of hardware, and the type of hardware is not limited. For example, the processor may be configured using hardware, such as a central processing unit (CPU), a micro processing unit (MPU), a programmable logic device, such as a field programmable gate array (FPGA), a dedicated circuit that is used to execute specific processing, such as an application-specific integrated circuit (ASIC), a graphic processing unit (GPU), or a neural processing unit (NPU). In addition, the type of hardware may be a combination of different types of hardware. In a case where a plurality of pieces of hardware are configured to execute one or more processes of a certain processor, the plurality of pieces of hardware may be present in devices physically separated from each other or may be present in the same device. Additionally, in any of the embodiments, the order of each process by the processor is not limited to the order described above and may be changed as appropriate. The hardware is configured using an electrical circuit (circuitry) in which circuit elements, such as semiconductor elements, are combined, or the like.

Further, the program may be software, such as firmware or a microcode. In addition, the program may be, for example, a program module group, and each function thereof may be implemented by a processor configured to execute the corresponding function. The program may be a program code or a plurality of code segments stored in one or more non-transitory computer-readable media (for example, storage media, other storages, or the like). The program may be distributed and stored across a plurality of non-transitory computer-readable media that are present in devices physically separated from each other. The program code or code segments may represent any combination of procedures, functions, subprograms, routines, subroutines, modules, software packages, classes, or commands, data structures, or program statements. The program code or code segment may be connected to another code segment or a hardware circuit by transmitting and receiving information, data, an argument, a parameter, or contents of a memory.

Additionally, in the above-described embodiment, an aspect has been described in which the information processing program 12 is stored (installed) in advance in the storage 13, but the present disclosure is not limited to this aspect. The information processing program 12 may be provided in a form recorded on a recording medium, such as a compact disc read-only memory (CD-ROM), a digital versatile disc read-only memory (DVD-ROM), and a universal serial bus (USB) memory. Alternatively, the information processing program 12 may be provided in a form that can be downloaded from an external device via a network.

The technology of the present disclosure extends to all kinds of program products. The program product includes all forms of products for providing a program. For example, the program product includes a program provided through a network such as the Internet, a non-transitory computer-readable recording medium, such as a CD-ROM, a DVD, and a USB memory in which the program is stored, and the like.

Hereinafter, the supplementary claims of the present disclosure will be described.

### Supplementary Claim 1

An information processing apparatus comprising:
a storage unit configured to store a plurality of pieces of calibration data for material discrimination, the plurality of pieces of calibration data being acquired by measuring a plurality of types of calibration members, each consisting of a combination of two or more types of base materials having different compositions, using a photon-counting detector that converts incident radiation into a detected photon count for each of a plurality of energy bins, the number of which is three or more, the plurality of pieces of calibration data representing energy spectra for combinations of the calibration members; and
a processor,
in which the processor is configured to:
   acquire projection data for each of the plurality of energy bins, the projection data being acquired by detecting radiation transmitted through a subject using the photon-counting detector;
   specify first matching calibration data that matches an energy spectrum of the projection data from among the plurality of pieces of calibration data;
   determine presence or absence of a singularity in an attenuation coefficient based on the projection data, based on a difference, for each of the plurality of energy bins, between an attenuation coefficient based on the first matching calibration data and the attenuation coefficient based on the projection data; and
   specify, in a case where determination is made that the singularity is present, second matching calibration data that matches the projection data, from among the plurality of pieces of calibration data, based on an energy spectrum of the projection data on a higher-energy side or a lower-energy side of the singularity.

### Supplementary Claim 2

The information processing apparatus according to Supplementary claim 1,
in which the processor is configured to specify, using a maximum likelihood estimation method, calibration data that matches the energy spectrum of the projection data from among the plurality of pieces of calibration data.

### Supplementary Claim 3

The information processing apparatus according to Supplementary claim 1 or 2,
in which the processor is configured to determine that the singularity is present in a case where a difference, for each of the plurality of energy bins, between an attenuation coefficient based on the specified calibration data and the attenuation coefficient based on the projection data is equal to or greater than a predetermined threshold value.

### Supplementary Claim 4

The information processing apparatus according to any one of Supplementary claims 1 to 3,
in which the processor is configured to:
   derive a material discrimination image based on the first matching calibration data in a case where determination is made that the singularity is not present; and
   derive a material discrimination image based on the second matching calibration data in a case where determination is made that the singularity is present.

### Supplementary Claim 5

An information processing method in an information processing apparatus including a storage unit configured to store a plurality of pieces of calibration data for material discrimination, the plurality of pieces of calibration data being acquired by measuring a plurality of types of calibration members, each consisting of a combination of two or more types of base materials having different compositions, using a photon-counting detector that converts incident radiation into a detected photon count for each of a plurality of energy bins, the number of which is three or more, the plurality of pieces of calibration data representing energy spectra for combinations of the calibration members, the information processing method comprising:
causing a computer to execute:
   acquiring projection data for each of the plurality of energy bins, the projection data being acquired by detecting radiation transmitted through a subject using the photon-counting detector;
   specifying first matching calibration data that matches an energy spectrum of the projection data from among the plurality of pieces of calibration data;
   determining presence or absence of a singularity in an attenuation coefficient based on the projection data, based on a difference, for each of the plurality of energy bins, between an attenuation coefficient based on the first matching calibration data and the attenuation coefficient based on the projection data; and
   specifying, in a case where determination is made that the singularity is present, second matching calibration data that matches the projection data, from among the plurality of pieces of calibration data, based on an energy spectrum of the projection data on a higher-energy side or a lower-energy side of the singularity.

### Supplementary Claim 6

An information processing program for causing a computer to function as an information processing apparatus including a storage unit configured to store a plurality of pieces of calibration data for material discrimination, the plurality of pieces of calibration data being acquired by measuring a plurality of types of calibration members, each consisting of a combination of two or more types of base materials having different compositions, using a photon-counting detector that converts incident radiation into a detected photon count for each of a plurality of energy bins, the number of which is three or more, the plurality of pieces of calibration data representing energy spectra for combinations of the calibration members, the information processing program causing the computer to execute:

a procedure of acquiring projection data for each of the plurality of energy bins, the projection data being acquired by detecting radiation transmitted through a subject using the photon-counting detector;

a procedure of specifying first matching calibration data that matches an energy spectrum of the projection data from among the plurality of pieces of calibration data;

a procedure of determining presence or absence of a singularity in an attenuation coefficient based on the projection data, based on a difference, for each of the plurality of energy bins, between an attenuation coefficient based on the first matching calibration data and the attenuation coefficient based on the projection data; and
a procedure of specifying, in a case where determination is made that the singularity is present, second matching calibration data that matches the projection data, from among the plurality of pieces of calibration data, based on an energy spectrum of the projection data on a higher-energy side or a lower-energy side of the singularity.

What is claimed is:

1. An information processing apparatus comprising:
a storage unit configured to store a plurality of pieces of calibration data for material discrimination, the plurality of pieces of calibration data being acquired by measuring a plurality of types of calibration members, each consisting of a combination of two or more types of base materials having different compositions, using a photon-counting detector that converts incident radiation into a detected photon count for each of a plurality of energy bins, the number of which is three or more, the plurality of pieces of calibration data representing energy spectra for combinations of the calibration members; and
a processor,
wherein the processor is configured to:
acquire projection data for each of the plurality of energy bins, the projection data being acquired by detecting radiation transmitted through a subject using the photon-counting detector;
specify first matching calibration data that matches an energy spectrum of the projection data from among the plurality of pieces of calibration data;
determine presence or absence of a singularity in an attenuation coefficient based on the projection data, based on a difference, for each of the plurality of energy bins, between an attenuation coefficient based on the first matching calibration data and the attenuation coefficient based on the projection data; and
specify, in a case where determination is made that the singularity is present, second matching calibration data that matches the projection data from among the plurality of pieces of calibration data, based on an energy spectrum of the projection data on a higher-energy side or a lower-energy side of the singularity.

2. The information processing apparatus according to claim 1,
wherein the processor is configured to specify, using a maximum likelihood estimation method, calibration data that matches the energy spectrum of the projection data from among the plurality of pieces of calibration data.

3. The information processing apparatus according to claim 1,
wherein the processor is configured to determine that the singularity is present in a case where a difference, for each of the plurality of energy bins, between an attenuation coefficient based on the specified calibration data and the attenuation coefficient based on the projection data is equal to or greater than a predetermined threshold value.

4. The information processing apparatus according to claim 2,
wherein the processor is configured to determine that the singularity is present in a case where a difference, for each of the plurality of energy bins, between an attenuation coefficient based on the specified calibration data and the attenuation coefficient based on the projection data is equal to or greater than a predetermined threshold value.

5. The information processing apparatus according to claim 1,
wherein the processor is configured to:
derive a material discrimination image based on the first matching calibration data in a case where determination is made that the singularity is not present; and
derive a material discrimination image based on the second matching calibration data in a case where determination is made that the singularity is present.

6. The information processing apparatus according to claim 2,
wherein the processor is configured to:
derive a material discrimination image based on the first matching calibration data in a case where determination is made that the singularity is not present; and
derive a material discrimination image based on the second matching calibration data in a case where determination is made that the singularity is present.

7. The information processing apparatus according to claim 3,
wherein the processor is configured to:
derive a material discrimination image based on the first matching calibration data in a case where determination is made that the singularity is not present; and
derive a material discrimination image based on the second matching calibration data in a case where determination is made that the singularity is present.

8. The information processing apparatus according to claim 4,
wherein the processor is configured to:
derive a material discrimination image based on the first matching calibration data in a case where determination is made that the singularity is not present; and
derive a material discrimination image based on the second matching calibration data in a case where determination is made that the singularity is present.

9. An information processing method in an information processing apparatus including a storage unit configured to store a plurality of pieces of calibration data for material discrimination, the plurality of pieces of calibration data being acquired by measuring a plurality of types of calibration members, each consisting of a combination of two or more types of base materials having different compositions, using a photon-counting detector that converts incident radiation into a detected photon count for each of a plurality of energy bins, the number of which is three or more, the plurality of pieces of calibration data representing energy spectra for combinations of the calibration members, the information processing method comprising:
causing a computer to execute:
acquiring projection data for each of the plurality of energy bins, the projection data being acquired by detecting radiation transmitted through a subject using the photon-counting detector;
specifying first matching calibration data that matches an energy spectrum of the projection data from among the plurality of pieces of calibration data;
determining presence or absence of a singularity in an attenuation coefficient based on the projection data, based on a difference, for each of the plurality of energy bins, between an attenuation coefficient based on the first matching calibration data and the attenuation coefficient based on the projection data; and
specifying, in a case where determination is made that the singularity is present, second matching calibration data that matches the projection data from among the plurality of pieces of calibration data, based on an energy spectrum of the projection data on a higher-energy side or a lower-energy side of the singularity.

10. A non-transitory computer-readable storage medium that stores an information processing program for causing a computer to function as an information processing apparatus including a storage unit configured to store a plurality of pieces of calibration data for material discrimination, the plurality of pieces of calibration data being acquired by measuring a plurality of types of calibration members, each consisting of a combination of two or more types of base materials having different compositions, using a photon-counting detector that converts incident radiation into a detected photon count for each of a plurality of energy bins, the number of which is three or more, the plurality of pieces of calibration data representing energy spectra for combinations of the calibration members, the information processing program causing the computer to execute:
a procedure of acquiring projection data for each of the plurality of energy bins, the projection data being acquired by detecting radiation transmitted through a subject using the photon-counting detector;
a procedure of specifying first matching calibration data that matches an energy spectrum of the projection data from among the plurality of pieces of calibration data;
a procedure of determining presence or absence of a singularity in an attenuation coefficient based on the projection data, based on a difference, for each of the plurality of energy bins, between an attenuation coefficient based on the first matching calibration data and the attenuation coefficient based on the projection data; and
a procedure of specifying, in a case where determination is made that the singularity is present, second matching calibration data that matches the projection data from among the plurality of pieces of calibration data, based on an energy spectrum of the projection data on a higher-energy side or a lower-energy side of the singularity.
